(19) United States
(12) Patent Application Publication
(10) Pub. No.: US 2019/0030373 A1
(43) Pub. Date: Jan. 31, 2019

(54) CHARGED PARTICLE BEAM TREATMENT SYSTEM

(71) Applicant: SUMITOMO HEAVY INDUSTRIES, LTD., Tokyo (JP)

(72) Inventors: Takuya Miyashita, Ehime (JP); Daizo Amano, Ehime (JP); Kazuya Taki, Kanagawa (JP)

(21) Appl. No.: 15/661,939

(22) Filed: Jul. 27, 2017

(51) Int. Cl. A61N 5/10; H05H 13/00

(52) U.S. Cl. CPC A61N 5/1067; A61N 5/1081; H05H 2277/11; A61N 2005/1095; A61N 2005/1087; H05H 13/005

(57) ABSTRACT

A charged particle beam treatment system includes a cyclotron that accelerates charged particles so as to emit a charged particle beam, an irradiation nozzle that irradiates a patient with the charged particle beam, a beam transport line along which the charged particle beam B emitted from the cyclotron is transported to the irradiation nozzle, profile monitors and that are provided in the beam transport line and detect a position of the beam, and steering electromagnets that are provided on an upstream side of the profile monitors, and adjust a position of the beam.

### BACKGROUND

### Technical Field

Certain embodiments of the present invention relates to a charged particle beam treatment system.

### Description of Related Art

The related art discloses a charged particle beam treatment system. The charged particle beam treatment system includes a cyclotron (particle accelerator) which accelerates ions and emits a photon beam, a beam transport line along which the photon beam emitted from the cyclotron is transported, and an irradiation device which irradiates an irradiation object with the photon beam transported along the beam transport line.

In this kind of charged particle beam treatment system, a position of a charged particle beam emitted from a cyclotron may change from day to day. In a case where a position of abeam is deviated relative to an expected position, there is concern that a beam may not transported to the expected position, and a currently transported beam may collide with a duct or the like so that an amount of a beam reaching an irradiation device is reduced.

### SUMMARY

According to the present invention, there is provided a charged particle beam treatment system including a cyclotron configured to accelerate charged particles so as to emit a charged particle beam; an irradiation device configured to irradiate an irradiation object with the charged particle beam; a beam transport line along which the charged particle beam emitted from the cyclotron is transported to the irradiation device; a beam position detection unit that is provided in the beam transport line and configured to detect a position of the passing charged particle beam; and a beam position adjustment unit that is provided on an upstream side of the beam position detection unit, and configured to adjust a position of the charged particle beam.

In the charged particle beam treatment system, it is possible to appropriately adjust a position of the charged particle beam with the beam position adjustment unit according to a position of the charged particle beam detected by the beam position detection unit.

The beam position detection unit may include a first detection unit configured to detect a position of the passing charged particle beam, and a second detection unit that is provided on a downstream side of the first detection unit, and configured to detect a position of the passing charged particle beam. According to this configuration, since passing positions of the charged particle beam are detected at two locations on an upstream side and a downstream side of the charged particle beam by the two detection units, it is possible to detect not only a passing position of the charged particle beam but also an advancing direction of the charged particle beam.

The charged particle beam treatment system according to the present invention may further include a degrader that is provided in the beam transport line, and configured to reduce and adjust the energy of the passing charged particle beam, the beam position detection unit may be provided on a downstream side of the degrader, and the beam position adjustment unit may be provided on an upstream side of the degrader.

If the charged particle beam has passed through the degrader, a beam diameter or the like changes due to divergence, but, in the system with the configuration, the beam position detection unit can detect a passing position of the charged particle beam after passing through the degrader. The beam adjustment unit adjusts a position of the charged particle beam on an upstream side of the degrader, and thus the charged particle beam can be made incident to a desired position in the degrader.

The charged particle beam treatment system according to the present invention may further include a degrader that is provided in the beam transport line, and configured to reduce and adjust the energy of the passing charged particle beam, and the beam position detection unit may include a particle detector configured to detect particles generated when the charged particle beam passes through the degrader, and a position calculation unit configured to detect a position where the particles detected by the particle detector are generated. According to this configuration, a passing position of the charged particle beam can be detected by using the degrader, and thus the degrader can also be used as a part of the beam position detection unit. The degrader is also used to irradiate an irradiation object with the charged particle beam, and can thus detect a passing position of the charged particle beam in real time even while the irradiation object is being irradiated with the charged particle beam.

The degrader may include a first damping member that reduces the energy of the passing charged particle beam, and a second damping member that is provided on a downstream side of the first damping member, and reduces the energy of the passing charged particle beam, and the particle detector may include a first particle detector configured to detect particles generated when the charged particle beam passes through the first damping member, and a second particle detector configured to detect particles generated when the charged particle beam passes through the second damping member. According to this configuration, since particles generated at positions of two damping members located on an upstream side and a downstream side of the charged particle beam, passing positions of the charged particle beam can be detected at two locations, and, as a result, it is possible to detect not only a position of the charged particle beam but also an advancing direction of the charged particle beam.

The beam position adjustment unit may include a first deflection unit configured to deflect the charged particle beam, and a second deflection unit that is provided on a downstream side of the first deflection unit, and configured to deflect the charged particle beam. According to this configuration, it is possible to adjust not only a position of the charged particle beam on a downstream side of the first and second deflection units but also an advancing direction thereof. As a result, it is possible to adjust an advancing direction of the charged particle beam to an appropriate direction (for example, an extending direction of a beam duct). Advantageous Effects of Invention

It is possible to provide a charged particle beam treatment system capable of adjusting a position of a charged particle beam emitted from a cyclotron to an appropriate position.

### BRIEF DESCRIPTION OF THE DRAWINGS

FIG. 1 is a diagram illustrating a charged particle beam treatment system according to one embodiment.

FIG. 2 is a diagram illustrating a profile monitor.

FIG. 3 is a schematic view in which a steering electromagnet and the profile monitor are viewed from a Y direction.

FIG. 4 is a flowchart illustrating adjustment of a beam position in the charged particle beam treatment system.

FIG. 5 is a diagram illustrating a charged particle beam treatment system according to another embodiment.

FIG. 6 is a diagram illustrating a degrader and a degrader monitor in another embodiment.

### DETAILED DESCRIPTION

It is desirable to provide a charged particle beam treatment system capable of adjusting a position of a charged particle beam emitted from a cyclotron to an appropriate position.

### One Embodiment

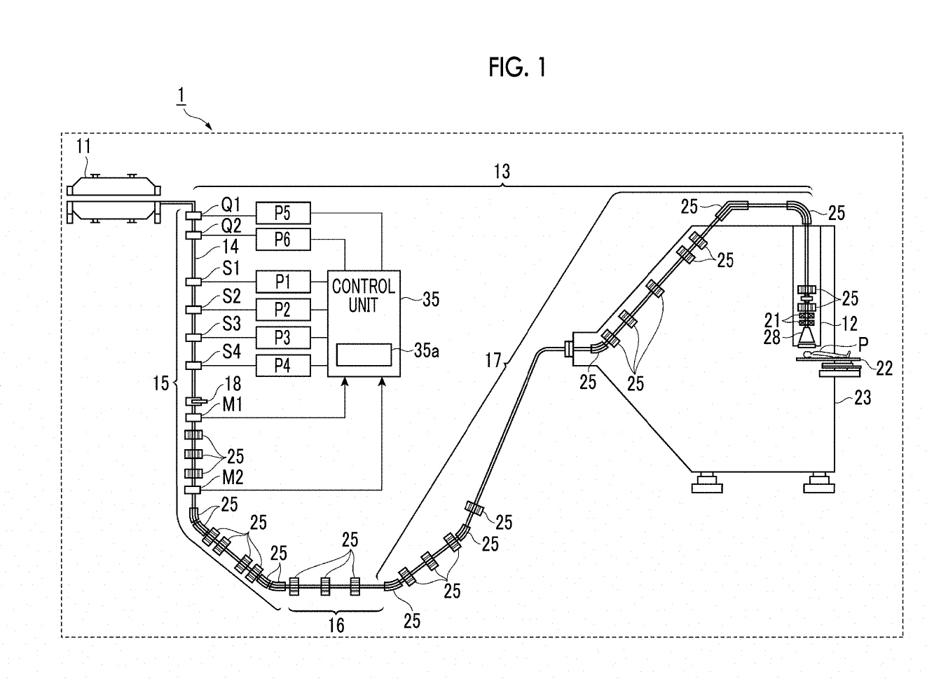

Hereinafter, with reference to FIG. 1, a charged particle beam treatment system 1 according to one embodiment will be described in detail. The terms “upstream” and “downstream” respectively indicate an upstream side (accelerator side) and a downstream side (patient side) of an emitted charged particle beam. On a plane which is orthogonal to a transport direction of a charged particle beam, predetermined one direction (horizontal direction) is set as an X direction, and a direction (for example, a vertical direction) which is orthogonal to the X direction is set as a Y direction. The beam transport direction is set as a Z direction.

The charged particle beam treatment system 1 illustrated in FIG. 1 is an apparatus used for cancer therapy or the like using radiotherapy, and includes a cyclotron 11 as an accelerator which accelerates charged particles so as to emit a charged particle beam, an irradiation nozzle 12 (irradiation device) which irradiates an irradiation object with the charged particle beam, and a beam transport line 13 along which the charged particle beam emitted from the cyclotron 11 is transported to the irradiation nozzle 12. The charged particle beam treatment system 1 further includes a degrader 18 which is provided in the beam transport line 13, and reduces energy of a charged particle beam so as to adjust a range of the charged particle beam, and a plurality of electromagnets 25 provided in the beam transport line 13.

In the charged particle beam treatment system 1, a tumor (irradiation object) of a patient P on a treatment table 22 is irradiated with a charged particle beam emitted from the cyclotron 11. The charged particle beam is obtained by accelerating particles with electric charge at a high speed, and includes, for example, a photon beam and a heavy particle (heavy ion) beam.

The irradiation nozzle 12 is provided inside a rotation gantry 23 which can be rotated around the treatment table 22 by 360 degrees, and can be moved to any rotation position by the rotation gantry 23. The irradiation nozzle 12 includes the electromagnets 25, scanning electromagnets 21, and a vacuum duct 28. The scanning electromagnets 21 are provided inside the irradiation nozzle 12. Each of the scanning electromagnets 21 includes an X-direction scanning electromagnet which performs scanning with a charged particle beam in the X direction on a plane which is orthogonal to an irradiation direction of the charged particle beam, and a Y-direction scanning electromagnet which performs scanning with the charged particle beam in the Y direction intersecting the X direction on a plane intersecting the irradiation direction of the charged particle beam. The charged particle beam applied by the scanning electromagnet 21 is deflected in the X direction and/or the Y direction, and thus the vacuum duct 28 on the downstream side of the scanning electromagnet has a diameter increasing toward the downstream side.

The beam transport line 13 includes a beam duct 14 through which a charged particle beam passes. The inside of the beam duct 14 is maintained in a vacuum state, and thus charged particles forming a currently transported charged particle beam are prevented from scattering due to air or the like. The electromagnets 25 provided on the beam transport line 13 include convergence electromagnets which cause a charged particle beam to converge and deflection electromagnets which deflect the beam.

The beam transport line 13 includes an energy selection system (ESS) 15 which selectively extracts a charged particle beam having an energy width smaller than a predetermined energy width from charged particle beams which have the predetermined energy width and are emitted from the cyclotron 11, abeam transport system (BTS) 16 which transports the charged particle beam having the energy width selected by the ESS 15 in a state in which energy is maintained, and a gantry transport system (GTS) 17 which transports the charged particle beam from the BTS 16 to the rotation gantry 23.

The degrader 18 reduces the energy of a passing charged particle beam so as to adjust a range of the charged particle beam. Since a depth from a body surface of a patient to a tumor which is an irradiation object differs for each patient, when a charged particle beam is applied to a patient, a range which is an arrival depth of the charged particle beam is required to be adjusted. The degrader 18 adjusts the energy of a charged particle beam which is emitted from the cyclotron 11 with predetermined energy, so that the charged particle beam appropriately reaches an irradiation object located at a predetermined depth in a patient' body. Such adjustment of charged particle beam energy in the degrader 18 is performed for each virtually sliced layer of an irradiation object.

The charged particle beam treatment system 1 includes two quadrupole electromagnets Q1 and Q2 provided directly on the downstream side of the cyclotron 11 in the beam transport line 13. The quadrupole electromagnet Q1 adjusts a width in the X direction of a currently transported charged particle beam according to a current supplied from an electromagnet power source P5. Similarly, the quadrupole electromagnet Q2 adjusts a width in the Y direction of the currently transported charged particle beam according to a current supplied from an electromagnet power source P6.

The charged particle beam treatment system 1 includes four steering electromagnets S1, S2, S3 and S4 (beam position adjustment unit) for adjusting a position of a beam in the X direction and the Y direction. The steering electromagnets S1 to S4 is provided in the beam transport line 13 between the quadrupole electromagnet Q1 and the degrader 18, and are arranged in the order of the steering electromagnets S1, S2, S3 and S4 from the upstream side toward the downstream side. The two steering electromagnets S1 and S3 of the four steering electromagnets function as a beam position adjustment unit which adjusts a beam position in the X direction, and the other two steering electromagnets S2 and S4 function as a beam position adjustment unit which adjusts a beam position in the Y direction.

Specifically, the steering electromagnet S1 (first deflection unit) deflects a beam in the X direction according to a current supplied from an electromagnet power source P1. In other words, an advancing direction of a beam passing through the steering electromagnet S1 is bent in the X direction by a magnetic field generating by the steering electromagnet S1. In this case, a bent angle of a beam is increased as a current supplied from the electromagnet power source P1 to the steering electromagnet S1 becomes larger, and a bent angle of the beam is reduced as the supplied current becomes smaller. A bent direction of a beam can be changed by changing positive and negative of a current supplied (a direction of a current supplied) from the electromagnet power source P1 to the steering electromagnet S1.

On the basis of the same configuration as that of the steering electromagnet S1, the steering electromagnet S3 (second deflection unit) deflects a beam in the X direction according to a current supplied from an electromagnet power source P3, the steering electromagnet S2 (first deflection unit) deflects a beam in the Y direction according to a current supplied from an electromagnet power source P2, and the steering electromagnet S4 (second deflection unit) deflects a beam in the Y direction according to a current supplied from an electromagnet power source P4.

The charged particle beam treatment system 1 includes two profile monitors M1 and M2 as beam position detection units which detect a passing position of a beam in the X direction and the Y direction. The profile monitor M1 (first detection unit) and the profile monitor M2 (second detection unit) are provided in the beam transport line 13 on the downstream side of the degrader 18, and the profile monitors M1 and M2 are arranged in this order from the upstream side toward the downstream side.

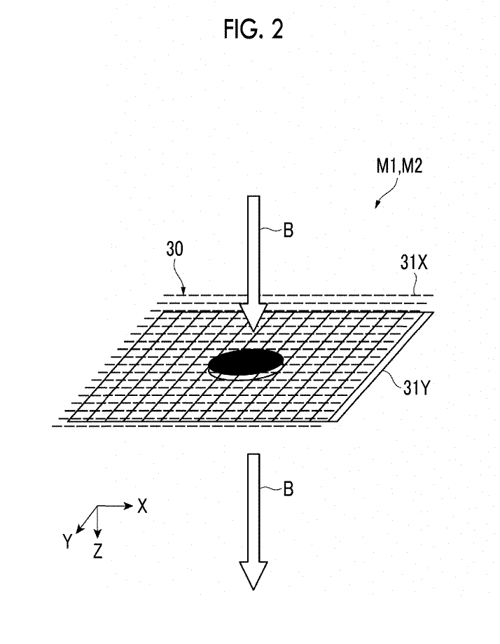

As illustrated in FIG. 2, the profile monitor M1 includes transmissive multi-strip wires 31X and 31Y built into an ionization chamber, and a high voltage is applied to the multi-strip wires 31X and 31Y. A plurality of (for example, 128) multi-strip wires 31X extending in the X direction and a plurality of (for example, 128) multi-strip wires 31Y extending in the Y direction are disposed to overlap each other in the Z direction, and form a wire grid 30 formed in a lattice shape when viewed from the Z direction. The wire grid 30 configured as mentioned above can indicate a position on an XY plane so as to correspond to each intersection (hereinafter, referred to as a “wire intersection”) between the multi-strip wires 31X and 31Y viewed from the Z direction.

When a beam B passes through the wire grid 30, electric charge is generated in the multi-strip wires 31X and 31Y irradiated with the beam B, and thus it is possible to detect a distribution of wire intersections included in an irradiation range of the beam B, that is, XY coordinates of an irradiation position (passing position) of the beam B by detecting the electric charge. The profile monitor M1 transmits an electrical signal indicating the XY coordinates of the passing position of the beam B to a control unit 35 which will be described later. The profile monitor M1 may detect a width of the beam in the X direction or a width of the beam in the Y direction on the basis of the distribution of the wire intersections included in the irradiation range. The profile monitor M2 has the same configuration as that of the profile monitor M1, and thus a repeated description will be omitted.

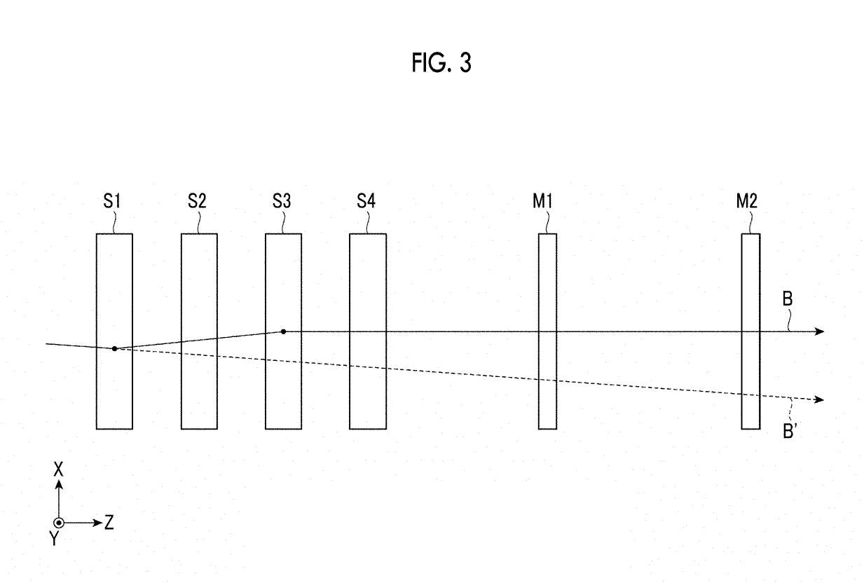

FIG. 3 is a schematic diagram in which the steering electromagnets S1 to S4, and the profile monitors M1 and M2 arranged in the beam transport line 13 are viewed from the Y direction. As described above, two steering electromagnets S1 and S3 arranged in the advancing direction of the beam B are provided to adjust a position of the beam B in the X direction. With this configuration, as illustrated in FIG. 3, the beam B can be bent twice in the X direction. Therefore, a bent angle of the beam B is adjusted by the steering electromagnets S1 and S3, and thus a position of the beam B in the X direction and the advancing direction in the X direction can be adjusted with respect to the beam B on the downstream side of the steering electromagnets S1 and S3. For example, in the example illustrated in FIG. 3, the beam B is bent upward in the figure in the X direction by the steering electromagnet S1, and is bent downward in the figure in the X direction by the steering electromagnet S3. Consequently, a position of the beam B is aligned with the center of the beam duct 14 (the center of the profile monitors M1 and M2), and the advancing direction of the beam B is parallel to the beam duct 14. B′ in the figure indicates a trajectory of the beam before adjustment.

Similarly, two steering electromagnets S2 and S4 arranged in the advancing direction of the beam B are provided to adjust a position of the beam B in the Y direction. With this configuration, the beam B can be bent twice in the Y direction. Therefore, a bent angle of the beam B is adjusted by the steering electromagnets S2 and S4, and thus a position of the beam B in the Y direction and the advancing direction in the Y direction can be adjusted with respect to the beam B on the downstream side of the steering electromagnets S2 and S4.

The two profile monitors M1 and M2 for detecting XY coordinates of a beam passing position are disposed in the transport direction of the beam B. With this configuration, it is possible to detect not only XY coordinates of a passing position of the beam B at each of two locations of the profile monitors M1 and M2 but also an X component and a Y component of the beam B in the advancing direction between the profile monitors M1 and M2.

The control unit 35 is formed of, for example, a computer, and transmits a control signal to each of the electromagnet power sources P1 to P6. Current parameters indicating currents to be supplied to the steering electromagnets S1 to S4 and the quadrupole electromagnets Q1 and Q2 are respectively added to the electromagnet power sources P1 to P6 by the control signals. Hereinafter, current parameters respectively added to the electromagnet power sources P1 to P6 by the control signals will be referred to as first to sixth current parameters in a differentiation manner, for example, a current parameter added to the electromagnet power source P1 is referred to as the “first current parameter”, and a current parameter added to the electromagnet power source P2 is referred to as the “second current parameter”.

The electromagnet power source P1 supplies a current corresponding to the first current parameter added by the control signal, to the steering electromagnet S1. The steering electromagnet S1 bends the beam B in a direction and at an angle corresponding to the supplied current as described above. As mentioned above, the control unit 35 adjusts a bent direction and a bent angle of the beam B in the steering electromagnet S1. Similarly, the control unit 35 adjusts bent directions and bent angles of the beam B in the steering electromagnets S2 to S4. Also similarly, the control unit 35 may adjust beam widths in the quadrupole electromagnets Q1 and Q2. As described above, the control unit 35 receives respective electrical signals indicating XY coordinates of passing positions of the beam B from the profile monitors M1 and M2.

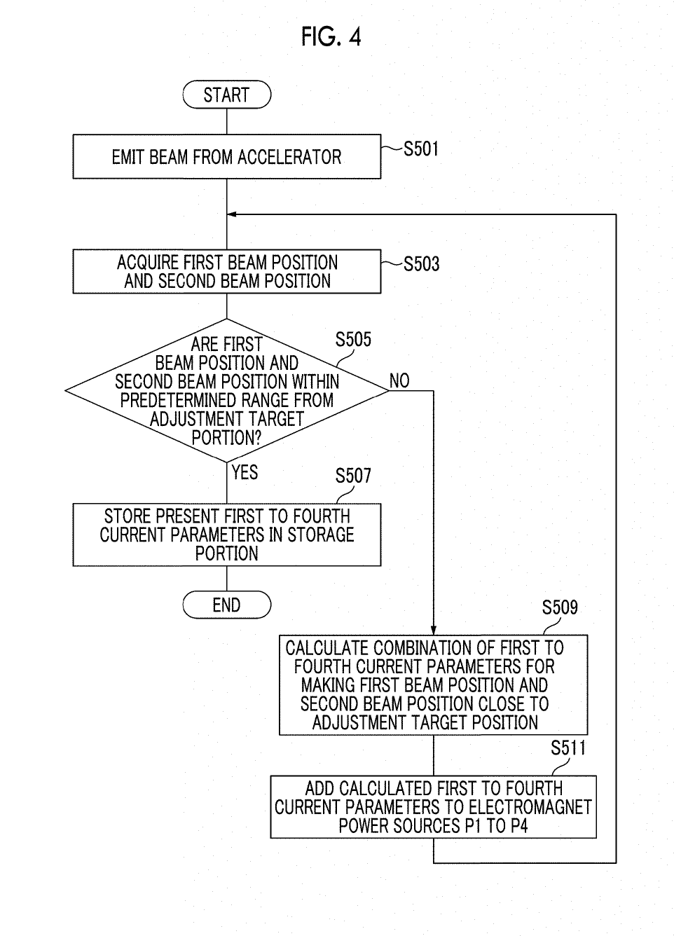

In the above-described charged particle beam treatment system 1, settings of current values in the steering electromagnets S1 to S4 are adjusted on the basis of passing positions of the beam B detected by the profile monitors M1 and M2, and thus a passing position and an advancing direction of the beam B on the downstream side of the steering electromagnets S1 to S4 are adjusted to appropriate positions. Hereinafter, with reference to FIG. 4, a description will be made of adjustment of a passing position and an advancing direction of the beam B in the charged particle beam treatment system 1. This adjustment process is performed, for example, about once a day prior to treatment of the patient P using the charged particle beam treatment system 1 every day. The adjustment process is automatically performed, for example, by the control unit 35 executing a program prepared in advance.

As illustrated in FIG. 4, in a state in which the charged particle beam B is emitted from the cyclotron 11 (step S501), each of the profile monitors M1 and M2 detects XY coordinates of a passing position of the beam B, and transmits an electrical signal to the control unit 35. The control unit 35 acquires the XY coordinates of the passing position (hereinafter, referred to as a “first beam position”) of the beam B in the profile monitor M1 and the XY coordinates of the passing position (hereinafter, referred to as a “second beam position”) of the beam B in the profile monitor M2 (step S503).

Next, the control unit 35 determines whether or not each of the first and second beam positions is within a predetermined range from an adjustment target position (step S505). For example, herein, in a case where an X coordinate of the first beam position, a Y coordinate of the first beam position, an X coordinate of the second beam position, and a Y coordinate of the second beam position are all within predetermined error ranges relative to coordinates of adjustment targets, “Yes” is determined, and, if otherwise, “No” is determined.

In a case where both of the first and second beam positions are within the predetermined ranges from the adjustment target position, setting has been performed so that a passing position of the beam B is located around the center of the beam duct 14, and an advancing direction of the beam B is substantially parallel to an extending direction of the beam duct 14.

In a case where Yes is determined in the above step S505, the control unit 35 stores the present first to fourth current parameters added to the electromagnet power sources P1 to P4 in a storage portion 35a of the control unit 35 (step S507), and finishes the process. Subsequently, the control unit 35 adds the first to fourth current parameters stored in the storage portion 35a to the electromagnet power sources P1 to P4, respectively.

On the other hand, in a case where No is determined in the above step S505, the flow proceeds to the next step S509. In step S509, the control unit 35 calculates, according to a predetermined algorithm, a combination of the first to fourth current parameters for making the X coordinate of the first beam position, the Y coordinate of the first beam position, the X coordinate of the second beam position, and the Y coordinate of the second beam position close to the coordinates of the adjustment targets (step S509). Next, the control unit 35 adds the first to fourth current parameters of the calculated combination to the electromagnet power sources P1 to P4, respectively, by using control signals (step S511). Thus, currents respectively corresponding to the first to fourth current parameters are supplied to the steering electromagnets S1 to S4 from the electromagnet power sources P1 to P4, and the steering electromagnets S1 to S4 bend the beam B in directions and at angles corresponding to the supplied currents. Next, the flow returns to the process in step S503, and the processes in step S503 and the subsequent steps are repeatedly performed. Thereafter, finally, “Yes” is determined in step S505, the first and second beam positions are within a predetermined position from the adjustment target position, the first to fourth parameters are stored in the storage portion 35a (step S507), and the process is finished.

Next, operations and effects of the above-described charged particle beam treatment system 1 will be described. In the charged particle beam treatment system 1, currents to be supplied to the electromagnet power sources P1 to P4 are set according to passing positions of the beam B detected by the profile monitors M1 and M2, and thus a passing position and an advancing direction of the beam B can be appropriately adjusted. The beam position adjustment process can be automatically performed by the control unit 35 according to a program which is prepared in advance, and thus time and effort for adjustment can be reduced compared with a method of manually adjusting settings of the steering electromagnets S1 to S4.

In the charged particle beam treatment system 1, on the basis of XY coordinates of the beam at two locations such as the profile monitors M1 and M2, a passing position and an advancing direction of the beam B in the X direction are adjusted by the steering electromagnets S1 and S3 at two locations, and a passing position and an advancing direction of the beam B in the Y direction are also adjusted by the steering electromagnets S2 and S4 at two locations. Therefore, passing positions and advancing directions of the beam B in the X direction and the Y direction can be appropriately adjusted, and thus it is possible to form a beam accurately advancing in the extending direction of the beam duct 14 around the center of the beam duct 14.

In this kind of charged particle beam treatment system, if the beam B has passed through the degrader 18, a diameter or the like of the beam B changes due to divergence. In contrast, in the charged particle beam treatment system 1, the profile monitors M1 and M2 are provided on the downstream side of the degrader 18, a passing position of the beam B after a change in a diameter or the like of the beam B is detected, and then a passing position and an advancing direction of the beam B is adjusted. As a result, the beam B whose position is appropriately adjusted reaches the irradiation nozzle 12. In the charged particle beam treatment system 1, since the steering electromagnets S1, S2, S3 and S4 are provided on the upstream side of the degrader 18, the beam B can be made to be accurately incident to a desired position in the degrader 18, and thus a range of the beam in the body of the patient P can also be accurately adjusted.

In the charged particle beam treatment system 1, the cyclotron 11 is used as an accelerator. The cyclotron 11 is greatly influenced by a change in a beam position at an outlet compared with other accelerators such as a synchrotron. Therefore, in the charged particle beam treatment system 1 using a cyclotron as an accelerator, the above-described configuration of automatically performing beam position adjustment is more appropriately used.

As described above, the profile monitors M1 and M2 may respectively detect a width of the beam B in the X direction and a width of the beam B in the Y direction. Therefore, the control unit 35 can also adjust widths of the beam B in the X direction and the Y direction by adjusting the fifth and sixth current parameters on the basis of the widths of the beam B detected by the profile monitors M1 and M2.

### Another Embodiment

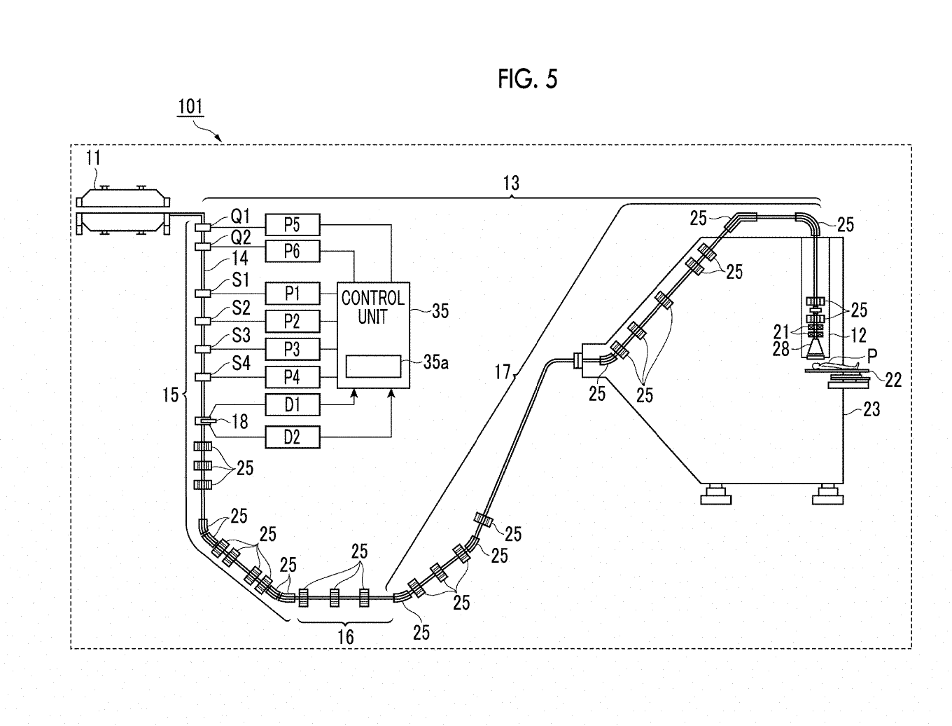

As illustrated in FIG. 5, a charged particle beam treatment system 101 of the present embodiment is different from the charged particle beam treatment system 1 in that degrader monitors D1 and D2 are provided instead of the profile monitors M1 and M2 (refer to FIG. 1). In the same manner as the profile monitors M1 and M2, the degrader monitors D1 and D2 function as beam position detection units detecting a beam position. Each of the degrader monitors D1 and D2 transmits an electrical signal indicating XY coordinates of a passing position of the beam B, to the control unit 35. Remaining configurations in the charged particle beam treatment system 101 are the same as those of the charged particle beam treatment system 1. Among constituent elements of the charged particle beam treatment system 101 of the present embodiment, constituent elements which are the same as or equivalent to those in one embodiment are given the same reference numerals on the drawings, and repeated description will be omitted.

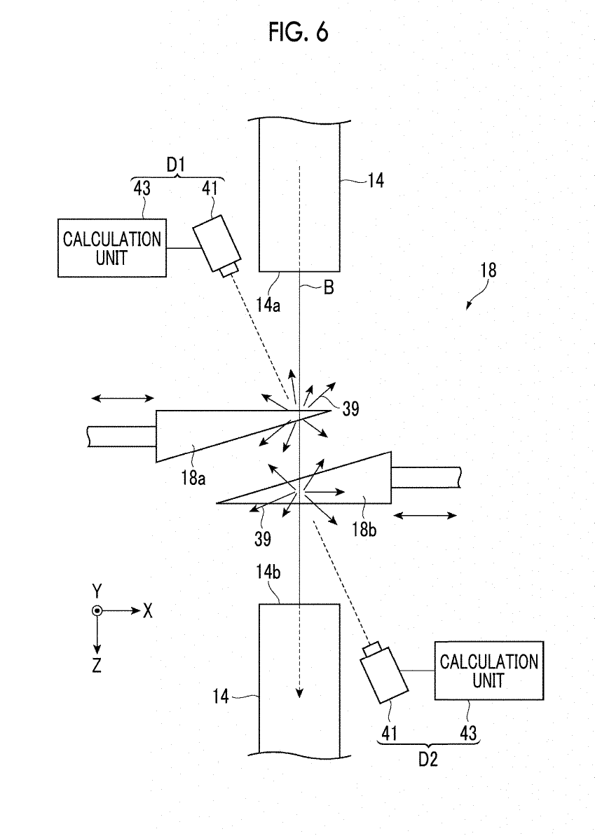

With reference to FIG. 6, a detailed description will be made of configurations of the degrader 18 and the degrader monitors D1 and D2. As illustrated in FIG. 6, the degrader 18 includes two damping members 18a and 18b arranged in the Z direction. Each of the damping members 18a and 18b has a wedge section which is sharpened in the X direction, and is disposed on a trajectory of the beam B. Of the damping members 18a and 18b, the damping member 18a (first damping member) is disposed on the upstream side, and the damping member 18b (second damping member) is disposed on the downstream side. The degrader 18 is provided at a location where the beam duct 14 is partially disconnected, and the damping members 18a and 18b are located outside the beam duct 14. In other words, the damping members 18a and 18b are disposed between a downstream section 14a of the beam duct 14 on the upstream side of the degrader 18 and an upstream section 14b of the beam duct 14 on the downstream side. The beam B emitted through the downstream section 14a from the beam duct 14 passes through the damping members 18a and 18b, and is introduced into the beam duct 14 again through the upstream section 14b.

When the beam B passes through the damping members 18a and 18b, the energy of the beam B is lost depending on thicknesses of the damping members 18a and 18b through which the beam is passing. The degrader 18 is provided with a driving mechanism (not illustrated) which moves the damping members 18a and 18b in a direction (for example, the X direction) of being inserted into and extracted from the trajectory of the beam B. Thicknesses of the damping members 18a and 18b through which the beam B passes are changed by inserting and extracting the damping members 18a and 18b into and from the trajectory of the beam B, and thus an amount of the energy of the beam B to be lost can be adjusted. As mentioned above, the energy of the beam B is adjusted by reducing the beam B to desired energy, and thus it is possible to adjust a range of the beam B in the body of the patient P.

Here, if the beam B passes through the degrader 18, secondary particles 39 are generated due to reaction between the beam B and the damping members 18a and 18b. The secondary particles 39 are emitted to the vicinities from passing locations of the beam B in the damping members 18a and 18b. The secondary particles 39 include, for example, gamma rays or electrons.

The degrader monitor D1 includes the damping member 18a, a camera 41 (first particle detector) which images the damping member 18a from an obliquely upstream side, and a calculation unit 43 (position calculation unit) which processes imaging data obtained by the camera 41. As the camera 41, a camera which can receive the secondary particles 39 and generate an image thereof is used. For example, a Compton camera, a camera having a scintillator, a PET camera, or a pinhole camera may be used as the camera 41. As described above, since the degrader 18 is provided at the location where the beam duct 14 is partially disconnected, the camera 41 can be provided outside the beam duct 14, and thus it is easy to secure an installation space for the camera 41.

The calculation unit 43 is, for example, a computer, performs predetermined processing calculation on the basis of imaging data obtained by the camera 41, and detects XY coordinates of a location where the secondary particles 39 are generated on the damping member 18a. The calculation unit 43 transmits an electrical signal indicating the XY coordinates of the location where the secondary particles 39 are generated on the damping member 18a, to the control unit 35. The location where the secondary particles 39 are generated on the damping member 18a is a passing position of the beam B at the position of the damping member 18a, and thus the control unit 35 treats the XY coordinates received from the calculation unit 43 as XY coordinates of the passing position of the beam B.

Instead of using an individual computer or the like, the calculation unit 43 may be included in the control unit 35 as one function of the control unit 35. The damping members 18a and 18b may be coated with a fluorescent substance which reacts with the secondary particles 39 so as to become fluorescent, and thus generates visible light. In this case, a camera (a typical visible light camera) which receives visible light and generates an image may be used as the camera 41. As the fluorescent substance, for example, a fluorescent coating material containing alumina or a fluorescent coating material containing silver and ZnS may be used.

In the same manner as degrader monitor D1, the degrader monitor D2 includes the damping member 18b, a camera 42 (second particle detector), and a calculation unit 44 (position calculation unit). The camera 42 of the degrader monitor D2 images the damping member 18b from an obliquely downstream side. The calculation unit 44 of the degrader monitor D2 transmits an electrical signal indicating the XY coordinates of the location where the secondary particles 39 are generated on the damping member 18b, to the control unit 35. Since the camera 42 has the same configuration as that of the camera 41, and the calculation unit 43 has the same configuration as that of the calculation unit 44, repeated description will be omitted.

Operations and effects of the charged particle beam treatment system 101 will be described.

In the charged particle beam treatment system 101, since the degrader monitors D1 and D2 are used instead of the above-described profile monitors M1 and M2, a position of the beam B can be detected at two positions of the damping member 18a and the damping member 18b disposed on the downstream side thereof, currents for the steering electromagnets S1 to S4 can be controlled on the basis of the detected position of the beam B, and thus a position of the beam B can be adjusted to an adjustment target position. In other words, the control unit 35 may treat a passing position of the beam B detected by the degrader monitor D1 as the above-described first beam position, and may treat a passing position of the beam B detected by the degrader monitor D2 as the above-described second beam position. Therefore, the same operations and effects as those of the charged particle beam treatment system 1 of one embodiment can be achieved.

While the patient P is irradiated with a beam by the charged particle beam treatment system 101 (during treatment of the patient P), the degrader 18 causes the beam B to pass therethrough so as to reduce the energy of the beam B, and the above-described secondary particles 39 are also generated from the damping members 18a and 18b during treatment of the patient P. Therefore, it is possible to acquire a position (XY coordinates) of the beam B from the degrader 18 by using the degrader monitors D1 and D2 without influencing the treatment. As mentioned above, since a passing position of the beam B can be detected in real time during treatment of the patient P, a process of performing feedback control on currents for the steering electromagnets S1 to S4 can be performed in real time during treatment on the basis of the detected passing position of the beam B. Therefore, it is possible to perform a process of controlling a passing position and an advancing direction of the beam B to be close to adjustment targets in real time during treatment, and, finally, the accuracy of a position of a beam applied to the patient P is also improved.

The present invention may be implemented in various forms to which various modifications and alterations are applied on the basis of only the above-described embodiments but also knowledge of a person skilled in the art. Modification examples may be configured by using the technical matter described in the above embodiments. An appropriate combination between the configurations of the respective embodiments may be used.

For example, in the embodiments, two steering electromagnets S1 and S3 are provided to perform beam position adjustment in the X direction, but the number of beam position adjustment units in the X direction may be one. Similarly, in the embodiments, two steering electromagnets S2 and S4 are provided to perform beam position adjustment in the Y direction, but the number of beam position adjustment units in the Y direction may be one. In the embodiments, two profile monitors M1 and M2 or two degrader monitors D1 and D2 are provided to detect a beam position at two locations, but the number of beam position detection units may one. In the present invention, a positional relationship in which the beam position detection unit is provided on the downstream side of the beam position adjustment unit may be provided, and, as in one embodiment, the configuration in which the degrader 18 is disposed between the beam position adjustment unit (steering electromagnets S1 and S2) and the beam position detection unit (profile monitors M1 and M2) is not essential.

It should be understood that the invention is not limited to the above-described embodiment, but may be modified into various forms on the basis of the spirit of the invention. Additionally, the modifications are included in the scope of the invention.

What is claimed is:

1. A charged particle beam treatment system comprising:
a cyclotron configured to accelerate charged particles so as to emit a charged particle beam;
an irradiation device configured to irradiate an irradiation object with the charged particle beam;
a beam transport line along which the charged particle beam emitted from the cyclotron is transported to the irradiation device;
a beam position detection unit that is provided in the beam transport line and configured to detect a position of the passing charged particle beam; and
a beam position adjustment unit that is provided on an upstream side of the beam position detection unit, and configured to adjust a position of the charged particle beam.

2. The charged particle beam treatment system according to claim 1,
wherein the beam position detection unit includes
a first detection unit configured to detect a position of the passing charged particle beam, and
a second detection unit that is provided on a downstream side of the first detection unit, and configured to detect a position of the passing charged particle beam.

3. The charged particle beam treatment system according to claim 1, further comprising:
a degrader that is provided in the beam transport line, and configured to reduce and adjust the energy of the passing charged particle beam,
wherein the beam position detection unit is provided on a downstream side of the degrader, and
wherein the beam position adjustment unit is provided on an upstream side of the degrader.

4. The charged particle beam treatment system according to claim 1, further comprising:
a degrader that is provided in the beam transport line, and configured to reduce and adjust the energy of the passing charged particle beam,
wherein the beam position detection unit includes
a particle detector configured to detect particles generated when the charged particle beam passes through the degrader, and
a position calculation unit configured to detect a position where the particles detected by the particle detector are generated.

5. The charged particle beam treatment system according to claim 4,
wherein the degrader includes
a first damping member that reduces the energy of the passing charged particle beam, and
a second damping member that is provided on a downstream side of the first damping member, and reduces the energy of the passing charged particle beam, and
wherein the particle detector includes
a first particle detector configured to detect particles generated when the charged particle beam passes through the first damping member, and
a second particle detector configured to detect particles generated when the charged particle beam passes through the second damping member.

6. The charged particle beam treatment system according to claim 2,
wherein the beam position adjustment unit includes
a first deflection unit configured to deflect the charged particle beam, and
a second deflection unit that is provided on a downstream side of the first deflection unit, and configured to deflect the charged particle beam.
